(19) United States
(12) Patent Application Publication
(10) Pub. No.: US 2026/0248328 A1
(43) Pub. Date: Aug. 27, 2026

(54) Portable Towel Warming Apparatus with Enhanced Heat Distribution and Thermal Insulation

(71) Applicant: J. Henry Scott, Boca Raton, FL (US)

(72) Inventor: J. Henry Scott, Boca Raton, FL (US)

(21) Appl. No.: 19/060,748

(22) Filed: Feb. 23, 2025

(51) Int. Cl. A47K 10/06; A61M 21/00; A61M 21/02; F24H 3/02; F24H 9/20; F24H 15/128; H05B 1/02

(52) U.S. Cl. CPC A47K 10/06; A61M 21/02; F24H 3/02; F24H 9/2071; F24H 15/128; H05B 1/0225; H05B 1/0272; A61M 2021/0016; A61M 2021/0066; A61M 2205/362; A61M 2205/3633

(57) ABSTRACT

A portable towel warming apparatus designed to efficiently and safely heat textiles, including towels, robes, blankets, and similar items. The apparatus comprises a housing that forms a warming chamber, insulated walls to minimize heat loss and maintain external surface safety, and a heating element configured for rapid and uniform heat distribution. Functional legs elevate the housing, preventing overheating of the supporting surface and promoting air circulation. The device features a control system with a timer, automatic shutoff functionality, and overheat protection for enhanced safety and convenience. Insulated walls retain heat for up to one hour post-operation, ensuring prolonged warmth of textiles. Optional features include forced-air circulation, wireless connectivity, and compartments for aromatic or therapeutic agents. The compact, portable design and robust safety features make the apparatus suitable for residential, commercial, and institutional applications.

### BACKGROUND OF THE INVENTION

### Field of Invention

The present invention relates to portable warming devices, and more particularly, to an apparatus designed for efficiently warming textiles such as towels, robes, blankets, and similar items. The invention incorporates features for optimized heat distribution and safety mechanisms, including insulated walls and a functional support structure to prevent overheating and enhance user convenience.

### Brief Summary of the Invention

The present invention provides a portable towel warming apparatus capable of rapidly warming textiles such as towels, robes, blankets, and similar items. The device features an integrated heating system that ensures even heat distribution across the warming chamber. The apparatus includes insulated walls to maintain warmth for up to one hour after automatic shutoff, thereby enhancing energy efficiency and user convenience. Functional legs are incorporated into the design to elevate the apparatus, aiding in heat dissipation and preventing overheating of the surface on which the unit is placed. This innovative combination of features ensures rapid and safe warming of textiles while maintaining user safety and comfort.

### BRIEF DESCRIPTION OF THE FIGURES

FIG. 1 illustrates a front view of the towel warming apparatus, shown from a top-down angle. The figure highlights the housing, warming chamber, and functional legs, as well as the arrangement of key external features such as the control panel and insulated walls.

FIG. 2 provides a back view of the towel warming apparatus, shown from a bottom-up angle. This figure emphasizes the functional legs, including their configuration and placement, as well as additional details of the apparatus's rear structural components and ventilation system.

### DETAILED DESCRIPTION

The present invention pertains to a portable towel warming apparatus designed to provide efficient and safe heating of textiles, including but not limited to towels, robes, blankets, and pajamas. The apparatus is constructed with an emphasis on rapid heating, energy efficiency, user safety, and operational convenience. Its design incorporates advanced thermal technology and functional structural elements to ensure optimal performance in residential, commercial, and institutional settings.

The apparatus features an external housing that forms an enclosed or semi-enclosed warming chamber. This housing is fabricated from heat-resistant materials such as thermoplastics, metals, or composite materials, chosen for their durability and ability to endure extended heating cycles without compromising structural integrity. The housing is surrounded by insulated walls constructed from multi-layer thermal barriers, including polyurethane foam, aerogel composites, or vacuum-insulated panels. These walls are critical for minimizing heat loss and maintaining the external surface temperature at safe levels during operation. The warming chamber itself is dimensioned to accommodate a variety of textiles and is equipped with a perforated or slotted internal platform positioned above the heating element to facilitate free circulation of heated air.

Central to the operation of the apparatus is a heating system engineered to deliver uniform and rapid heat distribution throughout the warming chamber. The heating element, located beneath the platform, may utilize resistive heating technology, such as nichrome wire, or radiant heating methods, including ceramic or infrared panels. The heating system is configured to achieve a target temperature range of 100° F. to 160° F., ensuring that textiles are warmed effectively without risk of fiber damage. To enhance the uniformity of heat distribution, the internal surfaces of the warming chamber are coated with heat-conductive materials such as aluminum or copper. In some embodiments, a forced-air mechanism, comprising a low-noise, high-efficiency fan, is integrated to circulate warm air evenly throughout the chamber, thereby eliminating hotspots.

The apparatus is elevated by functional legs that provide both structural support and thermal management. These legs are fabricated from high-strength, heat-resistant materials and are designed to create an air gap between the apparatus and the supporting surface. This elevation prevents heat buildup on the surface below, reducing the risk of overheating and potential damage to sensitive materials such as wood or plastic. Furthermore, the legs promote airflow around the apparatus, enhancing heat dissipation and contributing to overall safety. Stability is ensured by non-slip pads or grips affixed to the base of the legs, allowing the apparatus to remain secure on a variety of surfaces, including smooth or uneven flooring.

To ensure prolonged heat retention, the insulated walls are designed to maintain the internal temperature of the warming chamber for an extended period even after the apparatus powers down. This feature enables the apparatus to keep textiles warm for up to one hour post-operation, providing users with convenience and energy efficiency. The apparatus is controlled via an electronic system that includes a timer and automatic shutoff functionality. The timer allows users to select operation durations ranging from 10 to 60 minutes, while the automatic shutoff ensures that the heating element is deactivated after the selected time period, enhancing safety and conserving energy. The electronic system is further equipped with overheat protection, utilizing a thermal cutoff switch or electronic thermostat to monitor the internal temperature and deactivate the heating element if necessary. User interaction with the apparatus is facilitated by an intuitive control panel featuring buttons, dials, or a digital touchscreen, with LED indicators or a digital display providing real-time feedback on operational status.

To address safety concerns, the apparatus incorporates multiple protective features. In addition to overheat protection and insulated walls, a child lock mechanism may be included to prevent accidental activation by children. The exterior surfaces of the apparatus are designed to remain cool to the touch during operation, reducing the risk of burns or other injuries. Stability is further reinforced by an anti-tip design, which may include weight-balancing features or sensors to ensure the apparatus remains upright during use.

The invention is adaptable to various embodiments to suit specific user needs. In one embodiment, the heating element may be modular and replaceable, allowing users to extend the lifespan of the apparatus. In another embodiment, the apparatus may feature wireless connectivity, enabling users to control and monitor the device remotely via a smartphone application. Additional embodiments may include compartments or accessories for infusing warmed textiles with fragrances or therapeutic agents, such as essential oils, enhancing the user experience.

This portable towel warming apparatus is suitable for a wide range of applications, including residential use for personal comfort, hospitality environments to enhance guest experiences, and commercial settings such as spas or gyms. Its compact and portable design allows for easy transport and storage, while its robust construction ensures reliability across various environments. The features and embodiments described herein are illustrative and not limiting, as modifications and variations may be made without departing from the scope and spirit of the invention as defined in the appended claims.

### Detailed Description of Figures

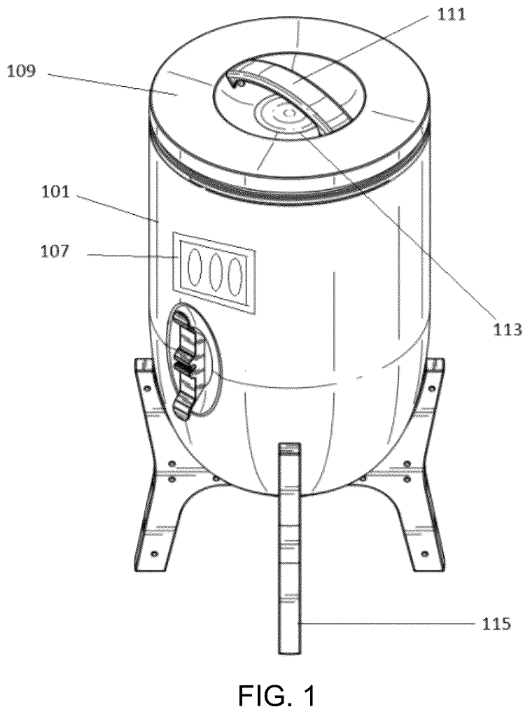

FIG. 1 illustrates a front perspective view of the portable towel warming apparatus, observed from a slightly elevated angle to provide a comprehensive visual understanding of the top-facing components. The external housing (101) is shown in its entirety, forming the warming chamber enclosed within insulated walls. The front-facing control panel (107) is prominently located on the housing, equipped with buttons or a touchscreen interface for adjusting operation settings, such as heating duration and intensity.

The top surface of the apparatus (109) features a smooth, heat-resistant design to ensure durability and safety during operation. Ergonomic carrying handles (111) or recessed grips are integrated along the edges of the top surface to facilitate portability. The perforated internal platform (113) can be partially visualized through the chamber opening, designed to optimize airflow within the chamber and ensure even heat distribution. The functional legs (115) are partially visible at the base, demonstrating the elevation of the unit above the supporting surface to enhance heat dissipation and safety.

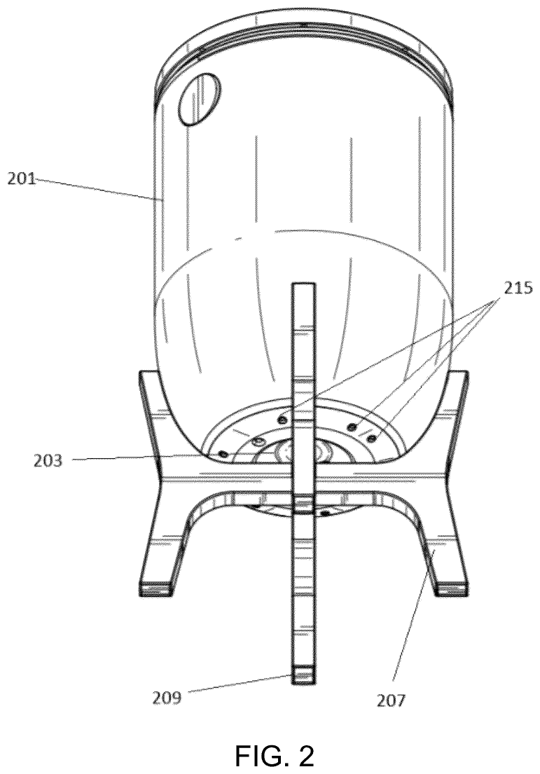

FIG. 2 depicts a rear perspective view of the apparatus from a slightly angled position below, highlighting structural and operational components typically concealed in standard views. The rear panel of the external housing (201) is visible, fabricated from heat-resistant and durable materials to provide structural integrity. The power cord inlet (203) is centrally located at the bottom of the rear panel, positioned for ease of connection to an electrical outlet while maintaining a clean aesthetic profile.

The base of the apparatus features four functional legs (207), each equipped with non-slip pads (209) to ensure stability on a variety of surfaces, including tile, wood, or carpeted flooring. The legs are spaced apart to allow for ventilation and airflow beneath the unit, preventing heat buildup on the supporting surface. The bottom surface of the warming chamber houses the heating element enclosure, designed to shield the heating components while enabling effective heat transfer to the warming chamber above. Ventilation grilles (215) are strategically placed near the base to facilitate airflow, ensuring optimal cooling of internal components and heat dissipation during operation.

What is claimed is:

1. A portable towel warming apparatus, comprising:
a. a housing forming a warming chamber;
b. a heating element disposed within the warming chamber, configured to generate heat for warming textiles;
c. insulated walls surrounding the warming chamber, the insulated walls being constructed to minimize heat loss and maintain a safe external surface temperature;
d. functional legs configured to elevate the housing above a supporting surface, the legs preventing overheating of the supporting surface and enhancing air circulation;
e. a control system operatively connected to the heating element, the control system including a timer and automatic shutoff functionality to regulate operation duration; and
f. a thermal protection system configured to deactivate the heating element upon detection of excessive temperature within the warming chamber.

2. The apparatus of claim 1, wherein the heating element comprises a resistive heating coil, an infrared heating panel, or a ceramic heating element.

3. The apparatus of claim 1, wherein the insulated walls are constructed from multi-layer thermal insulation materials, including polyurethane foam, aerogel composites, or vacuum-insulated panels.

4. The apparatus of claim 1, wherein the warming chamber further includes a perforated internal platform positioned above the heating element to facilitate the circulation of heated air.

5. The apparatus of claim 4, wherein the perforated internal platform is removable to accommodate larger textiles or for cleaning purposes.

6. The apparatus of claim 1, further comprising a forced-air mechanism, the mechanism including a fan configured to circulate heated air within the warming chamber to ensure uniform heat distribution.

7. The apparatus of claim 1, wherein the functional legs are fabricated from heat-resistant materials and include non-slip pads to ensure stability on various surfaces.

8. The apparatus of claim 1, wherein the control system further comprises a user interface including a digital display or LED indicators to provide real-time feedback on operational status.

9. The apparatus of claim 1, wherein the timer is adjustable, allowing the user to select operation durations ranging from 10 to 60 minutes.

10. The apparatus of claim 1, wherein the thermal protection system includes a thermal cutoff switch or electronic thermostat for monitoring and controlling the internal temperature of the warming chamber.

11. The apparatus of claim 1, wherein the insulated walls are configured to retain heat for up to one hour after the heating element has powered down, thereby maintaining the warmth of textiles.

12. The apparatus of claim 1, further comprising an anti-tip mechanism, the mechanism including weight-balancing features to ensure stability during operation.

13. The apparatus of claim 1, further comprising a compartment or accessory for holding aromatic or therapeutic agents, such as essential oils, for infusing warmed textiles with fragrances.

14. The apparatus of claim 1, wherein the warming chamber includes an interior surface coated with a heat-conductive material to facilitate even heat distribution.

15. The apparatus of claim 1, wherein the housing is equipped with ergonomic carrying handles or recessed grips to enhance portability.

16. The apparatus of claim 1, further comprising wireless connectivity, enabling remote control and monitoring of the apparatus via a smartphone application.

17. The apparatus of claim 1, wherein the functional legs include heat-dissipation fins to enhance cooling and prevent heat accumulation beneath the apparatus.

18. The apparatus of claim 1, wherein the apparatus is compact and portable, allowing for ease of storage and transport.

19. The apparatus of claim 1, wherein the housing is fabricated from a combination of thermoplastic, metal, or composite materials to ensure durability and heat resistance.

20. The apparatus of claim 1, wherein the warming chamber is configured to accommodate multiple textiles simultaneously, including towels, robes, blankets, and pajamas.
